(19) United States
(12) Patent Application Publication
(10) Pub. No.: US 2024/0024370 A1
(43) Pub. Date: Jan. 25, 2024

(54) PHARMACEUTICAL COMPOSITION INCLUDING ADIPOSE-DERIVED REGENERATIVE CELLS (ADRCS) FOR USE IN PREVENTION AND TREATMENT OF LIVER FIBROSIS OR LIVER CIRRHOSIS

(71) Applicant: CYTORI THERAPEUTICS K.K., Tokyo (JP)

(72) Inventor: Seijiro SHIRAHAMA, Tokyo (JP)

(73) Assignee: CYTORI THERAPEUTICS K.K., Tokyo (JP)

(21) Appl. No.: 18/256,866

(22) PCT Filed: Nov. 2, 2021
(86) PCT No.: PCT/JP2021/040342

§ 371 Date: Jun. 9, 2023

(30) Foreign Application Priority Data
Dec. 11, 2020 (JP) 2020-206130

(51) Int. Cl. A61K 35/35; A61P 1/16

(52) U.S. Cl. CPC A61K 35/35; A61P 1/16

(57) ABSTRACT

There is a pharmaceutical composition having adipose-derived regenerative cells (ADRCs) for use in prevention or treatment of liver fibrosis or liver cirrhosis. The composition is effective in maintaining and improving hepatic function and preventing and treating liver fibrosis or liver cirrhosis.

### CROSS-REFERENCE TO RELATED APPLICATIONS

This application is a National Stage application claiming priority from PCT Application No. PCT/JP 2021/040342, filed Nov. 2, 2021, which claims priority from Japanese Patent Application No. 2020-206130, filed Dec. 11, 2020, the disclosures of which are hereby incorporated by reference herein in their entireties.

### FIELD OF THE DISCLOSURE

The present disclosure relates to a pharmaceutical composition including adipose-derived regenerative cells (ADRCs) for use in prevention or treatment of liver fibrosis or liver cirrhosis.

### DESCRIPTION OF THE RELATED ART

Chronic liver disease involves, at its terminal stage, liver cirrhosis caused by progression of liver fibrosis associated with chronic inflammation, and hepatic failure associated therewith, further leading to the onset of liver cancer (Amico, G. D. et al. Natural history and prognostic indicators of survival in cirrhosis: A systematic review of 118 studies. 44, 217-231 (2006)).

When chronic liver disease is caused by virus such as viral hepatitis, complete extermination of the virus responsible therefor is the biggest goal of treatment. However, at present, it is impossible to achieve the complete extermination of hepatitis viruses in all cases even by the current most effective antiviral therapy using interferons, nucleic acid analog preparations, or directly acting antivirals. The cause of non-alcoholic steatohepatitis is still unknown, and an effective treatment method has yet to be established.

Chronic liver disease can progress into a liver cirrhosis condition, which can further progress into a hepatic failure condition which requires liver transplantation for life saving, or into a complication with liver cancer.

Liver transplantation has a drastic shortage of donors. Particularly, living liver transplantation is controversially invasive to donors. Furthermore, living liver transplantation requires expensive medical costs. As the number of indication cases of liver transplantation increases, medical economical burdens therefrom also become a major problem.

As for liver cancer which develops highly frequently in a liver cirrhosis condition, one of the major causes interfering with radical treatment thereof is reduction in hepatic reserve. In other words, if hepatic reserve can be improved, even liver cancer patients can receive radical treatment through surgical resection or radiofrequency ablation and thus be expected to have a better prognosis.

Against this backdrop, there is a demand for development of a treatment that prevents further worsening of hepatic functions in liver cirrhosis or liver fibrosis corresponding to the pre-stage of liver cirrhosis, and delays its progression into a terminal condition as much as possible.

On the other hand, mesenchymal stem cells (MSCs), which correspond to somatic stem cells, are known to have pluripotency to differentiate into adipocytes, chondrocytes, osteocytes, nerve cells, hepatocytes, and the like, and it has been reported that adipose-derived stem cells (ADSCs), which are rich in antigens of such MSCs-expressing cells, suppress persistent inflammation, which is the pathophysiological underlying process of liver cirrhosis, and restore liver function by regenerating and repairing damaged hepatocytes (Seki A, Sakai Y, Komura T, et al. Adipose tissue-derived stem cells as a regenerative therapy for a mouse steatohepatitis-induced cirrhosis model. Hepatology. 2013; 58: 1133-1142).

In addition, Japanese Patent Application publication No. 2018-1450% describes that adipose tissue-derived stromal cell population administered to the tail vein of mice reaches the site of liver tissue damage, and suggests that it is involved in the repair of liver tissue. However, Japanese Patent Application publication No. 2018-145096 only provides data based on a Concanavalin-A model (Example 2), a commonly used well-established model inducing acute, immune-mediated liver injury. The Concanavalin-A (ConA) model does not reflect chronic liver disease which involves the progressive destruction and regeneration of liver parenchyma leading to fibrosis and cirrhosis. Further, Heyman et al have reported that ConA and repeated applications are not feasible as a model of chronic injury as mice develop protective immune tolerance against ConA liver inflammation after successful clearance of the disease (Heymann et al., The concanavalin A model of acute hepatitis in mice, Laboratory animals 49 (S1) (2015) 12-20). It is also mentioned in Japanese Patent Application publication No. 2018-145096 that cells were delivered 1 hour after administration of ConA, the injuring agent. In this regard, Heymann et al., indicate in the paper that “DNA fragmentation and formation of apoptosis and increased serum transaminases can be typically observed as early as 5 h after ConA application”, and thus it can be said that delivery of cells at 1 hour after ConA administration was prior to development of any meaningful injury. As such, the data in Japanese Patent Application publication No. 2018-1450% provides no evidence of effectiveness in treating acute liver injury, and merely supports the prevention of acute liver injury at best. Moreover, the data in Japanese Patent Application publication No. 2018-1450% provides no evidence in the treatment or prevention of chronic liver injury, liver fibrosis and cirrhosis.

On the other hand, Claim 3 and the examples cited in Japanese Patent Application publication No. 2018-145096 discussed a specific mechanism of action by creation of liver tissue through the differentiation of hepatocytes. Not only has the inventors not demonstrated this action in chronic liver disease in said publication, but it completely differs from our present disclosure to treat chronic liver disease by boosting repair mechanisms such as angiogenesis and anti-fibrosis to maintain or restore function. In addition, although it has been stated in another report of Sakai et al., that adipose tissue-derived stromal cell population was administered to patients with liver cirrhosis based on different etiologies, sufficient data to confirm efficacy of such a cell population against chronic liver disease characteristic of liver fibrosis or liver cirrhosis has not been provided yet (Sakai Y, Tamura M, Seki A, et al., Phase I clinical study of liver regenerative therapy for cirrhosis by intrahepatic infusion of freshly isolated autologous adipose tissue—derived stromal/stem (regenerative) cell. Regenerative Therapy 6 (2017) 52-C4). There is still a need to establish effective treatments for chronic liver diseases characterized by liver fibrosis or cirrhosis.

### SUMMARY OF THE DISCLOSURE

An object of the present disclosure is to develop a novel pharmaceutical composition for prevention and treatment of liver fibrosis or liver cirrhosis.

The present inventors have confirmed that adipose-derived regenerative cells (ADRCs) are effective for maintenance of and improvement in hepatic functions in a liver cirrhosis condition, and completed a pharmaceutical composition for use in prevention and treatment of liver fibrosis or liver cirrhosis, using these ADRCs.

In one aspect, the present disclosure relates to a pharmaceutical composition including ADRCs for use in prevention or treatment of liver fibrosis or liver cirrhosis.

In another aspect, the present disclosure relates to a method for prevention or treatment of liver fibrosis or liver cirrhosis, including administrating ADRCs to a subject in a therapeutically effective amount, in which the therapeutically effective amount is an amount sufficient to cause a detectable improvement or maintenance of hepatic functions.

In at least one embodiment, the ADRCs of the present disclosure may be an arbitrary heterogeneous or homogeneous cell population containing one or more types of adipose-derived regenerative cells including adipose-derived stem cells (ADSCs), endothelial cells (including vascular and lymphatic endothelial cells), endothelial precursor cells, macrophages, fibroblasts, pericytes, smooth muscle cells, preadipocytes, keratinocytes, unipotent or multipotent precursor and progenitor cells (and progeny thereof), and lymphocytes.

In at least one embodiment, the ADRCs of the present disclosure may comprise ADSCs at a percentage of at least 0.1%, 0.2%, 0.3%, 0.4%, 0.5%, 0.6%, 0.7%, 0.8%, 0.9%, 1.0%, 2%, 3%, 4%, 5%, 10%, 15%, 20%, 25%, 50%, or more of all cellular components. The percentage is based on total number of nucleated cells found in the adipose-derived cell population.

In at least one embodiment, the ADRCs of the present disclosure may be isolated from adipose tissue and then used directly without being cultured before administration to a subject. In at least one embodiment, the ADRCs of the present disclosure are uncultured cells.

In at least one embodiment, the ADRCs of the present disclosure includes cryopreserved cells. In at least one embodiment, at least a portion of the ADRCs of the present disclosure can be cryopreserved for subsequent use, by using a method known to those skilled in the art.

The ADRCs of the present disclosure are cells collected from the adipose tissue of same or different animal species. In at least one embodiment, the ADRCs of the present disclosure can be cells collected from a patient's own adipose tissue, and at least one embodiment includes autologous subcutaneous adipose-derived regenerative cells, for circumventing immune rejection.

In at least one embodiment, characterization of the cell population containing ADRCs may be performed using a technique generally used in the art, for example, it may be performed by variously combining cell markers or gene markers.

In at least one embodiment, the ADRCs of the present disclosure include cell surface maker CD45-positive cells.

In some approaches, ADRCs are CD14⁺ or CD11b⁺.

In at least one embodiment, the ADRCs of the present disclosure are identified by expression of cell surface marker selected from the group consisting of CD34, CD44, CD45, CD90 and CD105.

In at least one embodiment, the ADRCS are formulated to be administered systemically, e.g., intravenously or intraarterially or via the lymphatic system. In at least one embodiment, the ADRCs are formulated to be administered locally, e.g., topically or by local injection.

In at least one embodiment, the method for prevention or treatment of liver fibrosis or liver cirrhosis, further including selecting, identifying, or classifying the subject who needs the above prevention or treatment is made by a physician or clinical or diagnostic evaluation. In more embodiment, said methods further comprise diagnosing said subject by image findings or histology.

In at least one embodiment, the subject may be a patient with liver fibrosis or liver cirrhosis caused by non-alcoholic steatohepatitis or fatty liver disease. In more specific embodiment, the patient with the above diseases may satisfy one or more the certain criteria including an amount of alcohol intake, a possible condition or complication responsible for fatty liver, selected from the group consisting of obesity, visceral fat, metabolic syndrome and diabetes.

In at least one embodiment, the pharmaceutical composition of the present disclosure including ADRCs are administered by the form of, for example, intraarterial, intravenous, intraportal, intradermal, subcutaneous, intramuscular or intraperitoneal injection, though the administration method is not limited thereto.

In at least one embodiment, administration by intraarterial, intravenous or intraportal injection is preferred, and hepatic artery administration is particularly preferably performed, because administered ADRCs reach the liver, an organ to be treated, through blood circulation.

In at least one embodiment, the ADRCs may formulated and administered in a cell density of least 1×10² cells/mL, at least 1×10³ cells/mL, at least 1×10⁴ cells/mL, at least 1×10⁵ cells/mL, at least 1×10⁶ cells/mL, at least 1×10⁷ cells/mL, at least 1×10⁸ cells/mL, at least 1×10⁹ cells/mL, or at least 1×10¹⁰ cells/mL in an isotonic electrolyte transfusion. In more specific embodiment, 1×10³ to 1×10⁹ cells/mL in an isotonic electrolyte transfusion, for example, a lactated ringer's solution is preferred. At least one embodiment includes a cell density of 1×10⁶ cells/mL. In a further embodiment, the infusion solution containing the ADRCs are administered at 3.3×10⁵ cells/kg×BW or more.

In certain embodiments, the pharmaceutical compositions of the present disclosure exhibit an improvement in serum albumin concentration and/or an improvement in prothrombin activity in the prevention or treatment of liver fibrosis and/or liver cirrhosis. Here, serum albumin concentration and prothrombin activity are generally known as indexes for evaluating liver function. In at least one embodiment, the pharmaceutical compositions of the present disclosure are a single-cell suspension of ADRCs which is suitable for administration by intra-arterial, intravenous or intraportal injection. Here, an appropriate enzyme reagent is used for removing cell masses and preparing a single-cell suspension which is suitable for said administration. Intravase® 840 (Cytori Therapeutics Inc.) may also be mentioned as an example of said enzyme reagent.

In certain embodiments, the pharmaceutical compositions of the present disclosure are a single-cell suspension of ADRCs which reduces the risk of vascular occlusion and subsequent complications by using a filter membrane screen to remove cell aggregates and debris prior to intravenous or intraarterial cell delivery to a patient. For the above purpose, any filter membrane screen having an appropriate pore size, for example, a macro syringe filter having an average pore size of 43 microns (Cytori Therapeutics Inc.) may be used.

Specifically, the present disclosure can be summarized as following embodiments. A pharmaceutical composition including adipose-derived regenerative cells for use in prevention or treatment of liver fibrosis or liver cirrhosis.

In at least one embodiment, the adipose-derived regenerative cells are a heterogeneous or homogeneous cell population containing one or more types of adipose-derived regenerative cells selected from adipose-derived stem cells (ADSCs), endothelial cells, endothelial precursor cells, macrophages, fibroblasts, pericytes, smooth muscle cells, preadipocytes, keratinocytes, unipotent or multipotent precursor and progenitor cells and progeny thereof, and lymphocytes.

In at least one embodiment, the adipose-derived regenerative cells include adipose-derived stem cells (ADSCs) at a proportion of at least 0.1% of all cellular components of the adipose-derived regenerative cells.

In at least one embodiment, the adipose-derived regenerative cells are uncultured cells.

In at least one embodiment, the adipose-derived regenerative cells are cryopreserved cells.

In at least one embodiment, the adipose-derived regenerative cells are autologous subcutaneous adipose-derived regenerative cells.

In at least one embodiment, the adipose-derived regenerative cells are positive for cell surface marker CD45.

In at least one embodiment, the liver fibrosis or liver cirrhosis is caused by a chronic liver injury.

In at least one embodiment, the liver fibrosis or liver cirrhosis is caused by non-alcoholic steatohepatitis.

In at least one embodiment, a patient diagnosed with liver cirrhosis caused by non-alcoholic steatohepatitis by image findings or histology satisfies the following criteria (1) to (3):
(1) an alcohol intake is 20 g or less per day in terms of ethanol consumption;
(2) other known causes of liver damage have not been identified; and
(3) the patient has at least one possible condition or complication responsible for fatty liver, selected from the group consisting of obesity, visceral fat, metabolic syndrome and diabetes.

In at least one embodiment, the liver fibrosis or liver cirrhosis is based on fatty liver disease.

In at least one embodiment, a patient diagnosed with liver cirrhosis based on fatty liver disease by image findings or histology, satisfies the following criteria (4) to (6):
(4) an alcohol intake is more than 20 g and 70 g or less per day in terms of ethanol consumption;
(5) other known causes of liver damage have not been identified; and
(6) the patient has at least one possible condition or complication responsible for fatty liver, selected from the group consisting of obesity, visceral fat, metabolic syndrome and diabetes.

In at least one embodiment, the pharmaceutical composition is prepared such that the pharmaceutical composition is administered by intra-arterial, intravenous or intraportal injection.

In at least one embodiment, the pharmaceutical composition is prepared in an amount of a cell density of 1×10³ to 1×10⁹ cells/mL.

In at least one embodiment, the treatment of the liver cirrhosis comprises improvement in serum albumin level or prothrombin activity.

In at least one embodiment, the pharmaceutical composition is prepared in the form of a single-cell suspension of ADRCs.

The pharmaceutical composition of the present disclosure including ADRCs brings about marked effects on maintenance of and improvement in hepatic functions and can thereby be used in prevention and treatment of liver fibrosis or liver cirrhosis, particularly, prevention and treatment of liver fibrosis or liver cirrhosis caused by non-alcoholic steatohepatitis (NASH) and liver fibrosis or liver cirrhosis based on fatty liver disease, for which any effective treatment method has not yet been established.

### BRIEF DESCRIPTION OF THE DRAWINGS

FIG. 1A shows a transition of serum albumin value.

FIG. 1B shows a transition of prothrombin activity.

FIG. 2A shows a transition of serum albumin value in average.

FIG. 2B shows a transition of serum albumin value individual.

FIG. 3A shows a transition of prothrombin activity in average.

FIG. 3B shows a transition of prothrombin activity individual.

FIG. 4A shows NAS (steatosis).

FIG. 4B shows NAS (lobular inflammation).

FIG. 5A shows NAS (hepatocyte ballooning).

FIG. 5B shows Matteoni classification.

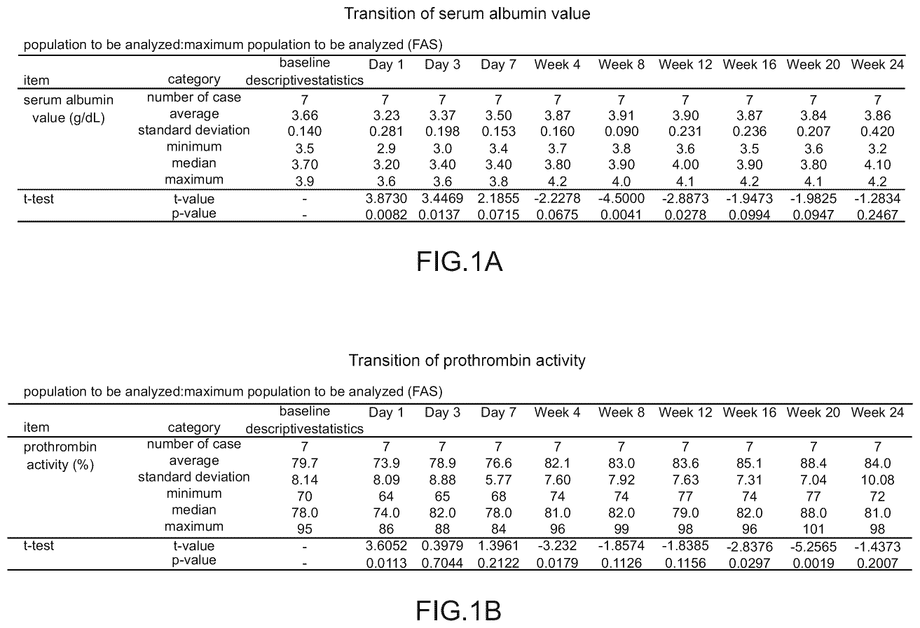

### DETAILED DESCRIPTION OF THE DISCLOSURE

### Definitions

As used herein, the term “about,” when referring to a stated numeric value, indicates a value within ±10% of the stated numeric value.

As used herein, the term “derived” means a form isolated, purified or separated from a certain subject. Thus, “adipose-derived regenerative cells” or “ADRCs” mean regenerative cells isolated, purified or separated from adipose tissue. Similarly, “adipose-derived stem cells” or “ADSCs” mean stem cells isolated, purified or separated from adipose tissue. The term “derived” does not encompass cells that are extensively cultured (e.g., placed in culture conditions in which the majority of dividing cells undergo 3, 4, 5 or less, cell doublings), from cells isolated directly from a tissue, e.g., adipose tissue, or cells cultured or expanded from primary isolates. Accordingly, “ADRCs” or “ADSCs” may be in their “native” form as separated from the adipose tissue matrix and exclude extensively cultured cells.

As used herein, “regenerative cells” refers to any heterogeneous or homologous cells obtained using the systems and methods of embodiments disclosed herein which cause or contribute to complete or partial regeneration, restoration, or substitution of structure or function of an organ, tissue, or physiologic unit or system to thereby provide a therapeutic, structural or cosmetic benefit. Examples of regenerative cells may include: adult stem cells (ASCs), endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes.

In some contexts, the term “progenitor cells” or “precursor cells” refers to a cell that is unipotent, bipotent, or multipotent with the ability to differentiate into one or more cell types, which perform one or more specific functions and which have limited or no ability to self-renew. Some of the progenitor cells disclosed herein may be pluripotent.

As used herein, cell is “positive” for a particular marker when that marker is detectable using a technique generally used in the art. For example, when the ADRCs of the present disclosure are positive for CD45, the term “positive” means that CD45 is detectable at a level greater than background (in comparison to, e.g., an isotype control or an experimental negative control for any given assay). A cell is also positive for a marker when that marker can be used to distinguish the cell from at least one other cell type, or can be used to select or isolate the cell when present or expressed by the cell.

As used herein, the term “adipose tissue” means a tissue containing multiple cell types including adipocytes and vascular cells. The adipose tissue includes multiple regenerative cell types, including adult stem cells (ASCs) and endothelial progenitor and precursor cells. The adipose tissue may mean fat, including the connective tissue that stores the fat.

In some contexts, the term “adipose tissue derived cells” refers to cells extracted from adipose tissue that has been processed to separate the active cellular component from the mature adipocytes and connective tissue. Separation may be partial or full. That is, the “adipose-derived cells” may or may not contain some adipocytes and connective tissue and may or may not contain some cells that are present in aggregates or partially disaggregated form (for example, a fragment of blood or lymphatic vessel including two or more cells that are connected by extracellular matrix). This fraction is also referred to herein as “adipose-derived cells” or “ADCs.” Typically, “adipose tissue-derived cells” refers to the pellet of cells obtained by washing and separating the cells from the adipose tissue. The pellet is typically obtained by centrifuging a suspension of cells so that the cells aggregate at the bottom of a centrifuge container, or alternatively concentrating the cells in a different manner.

(Indication)

The pharmaceutical compositions of the present disclosure including ADRCs can be used for the prevention or treatment of liver fibrosis or liver cirrhosis.

Liver failure and cirrhosis occur as a result of a variety of chronic hepatic injuries that share overlapping pathogenic processes including inflammation, hepatocyte necrosis, impaired regenerative capacity and liver fibrosis/cirrhosis.

Liver fibrosis, which also corresponds to a pre-stage of liver cirrhosis, is a common response to hepatocellular necrosis or injury, which may be induced by a wide variety of agents, e.g., any process disturbing hepatic homeostasis (especially inflammation, toxic injury, diabetes, steatohepatitis, or altered hepatic blood flow) and infections of the liver (viral, bacterial, fungal, and parasitic).

The normal liver is made up of hepatocytes and sinusoids distributed within an extracellular matrix composed of collagen (predominantly types I, III, and IV) and noncollagen proteins, including glycoproteins (e.g., fibronectin, laminin) and several proteoglycans (e.g., heparan sulfate, chondroitin sulfate, dermatan sulfate, hyaluronate).

Fibroblasts, normally found only in the portal tracts, can produce collagen, large glycoproteins, and proteoglycans. Other liver cells (particularly stellate cells, hepatocytes and fat-storing Kupffer, and endothelial cells) also can produce extracellular matrix components. Fat-storing cells, located beneath the sinusoidal endothelium in the space of Disse, are precursors of fibroblasts, capable of proliferating and producing an excess of extracellular matrix. The development of fibrosis from active deposition of collagen is a consequence of liver cell injury, particularly necrosis, and inflammatory cells. The precise factors released from these cells is not known, but one or more cytokines or products of lipid peroxidation are likely. Kupffer cells and activated macrophages produce inflammatory cytokines. New fibroblasts form around necrotic liver cells; increased collagen synthesis leads to scarring. Quiescent stellate cells can become activated leading to upregulation of fibrosis. Fibrosis may derive from active fibrogenesis and from impaired degradation of normal or altered collagen. Fat-storing cells, Kupffer cells, and endothelial cells are important in the clearance of type I collagen, several proteoglycans, and denatured collagens. Changes in these cells' activities may modify the extent of fibrosis. For the histopathologist, fibrous tissue may become more apparent from passive collapse and condensation of preexisting fibers.

Thus, increased synthesis or reduced degradation of collagen results in active deposition of excessive connective tissue, which affects hepatic function: (1) Pericellular fibrosis impairs cellular nutrition and results in hepatocellular atrophy. (2) Within the space of Disse, fibrous tissue accumulates around the sinusoids and obstructs the free, passage of substances from the blood to the hepatocytes. (3) Fibrosis around hepatic venules and the portal tracts disturbs hepatic blood flow. Venous resistance across the liver Increases from portal vein branches to sinusoids and finally to hepatic veins. All three routes can be involved.

The fibrous bands that link portal tracts with central veins also promote anastomotic channels: Arterial blood, bypassing the normal hepatocytes, is shunted to efferent hepatic veins, which further impairs hepatic function and can accentuate hepatocellular necrosis. The extent to which these processes are present determines the magnitude of hepatic dysfunction: e.g., in congenital hepatic fibrosis, large fibrous bands involve predominantly the portal regions but usually spare the hepatic parenchyma. Congenital hepatic fibrosis thus presents as portal hypertension with preserved hepatocellular function.

Non-alcoholic fatty liver disease (NAFLD) is a chronic liver disease characterized by hepatic fat accumulation in the absence of excessive alcohol consumption, and defined by the presence of steatosis in at least 5% of hepatocytes. NAFLD is a heterogeneous disease, including distinct histological conditions with different prognoses. Non-alcoholic fatty liver (NAFL) is defined as the presence of hepatic steatosis in at least 5% of the hepatocytes, without evidence of hepatocellular injury in the form of hepatocyte ballooning; non-alcoholic steatohepatitis (NASH) is defined as the presence of at least 5% hepatic steatosis and inflammation with hepatocyte injury (e.g., ballooning), with or without fibrosis. Both NAFL and NASH can progress to advanced fibrosis and cirrhosis, but the risk of progression is much greater in patients with NASH compared to NAFL, with a higher potential to advance to cirrhosis, liver failure and liver cancer.

In NASH, genetic susceptibility and poor eating habits predispose to the development of insulin resistance and hepatic steatosis. In this context, lipotoxic metabolites of saturated fatty acids can cause lipotoxicity, a process that leads to cellular damage through excessive oxidative stress. Damaged hepatocytes release damage endogenous associated molecular patterns that activate pro-inflammatory signaling pathways via toll-like receptors. Subsequent activation of Kupffer cells and inflammasome promote the massive release of pro-inflammatory, pro-fibrogenic cytokines and ligands. Hepatic stellate cells are then stimulated to produce high amounts of extracellular matrix leading to progressive fibrosis. Kupffer cell activation favors a pro-inflammatory microenvironment that triggers an adaptive immune response Th17-mediated.

Moreover, chronic portal inflammatory infiltrate boosts a ductular reaction and hepatic progenitor cells recruitment. All of these factors encourage progressive fibrosis that constitutes an imbalance between tissue injury and repair secondary to influence of various inflammatory cells.

As we mentioned the above, the pharmaceutical composition of the present disclosure can also be used for the prevention or treatment of liver fibrosis. The liver fibrosis also corresponds to a pre-stage of liver cirrhosis, and therefore a suppression or slow down in the progress of a liver fibrosis must be important to present a liver cirrhosis.

A disease as an indication of the pharmaceutical composition of the present disclosure includes liver cirrhosis based on various chronic liver diseases, for example, viral hepatitis type B, viral hepatitis type C, alcoholic liver disease, non-alcoholic steatohepatitis, fatty liver disease, autoimmune hepatitis or primary biliary cholangitis.

In at least one embodiment, the pharmaceutical composition of the present disclosure is administered to a patient diagnosed with liver cirrhosis based on non-alcoholic steatohepatitis or liver cirrhosis based on fatty liver disease.

In certain embodiment, the patient diagnosed with liver cirrhosis based on non-alcoholic steatohepatitis by image findings or histology may satisfy the following criteria (1) to (3):
(1) an alcohol intake is 20 g or less per day in terms of ethanol consumption;
(2) other known causes of liver damage have not been identified; and
(3) the patient has at least one possible condition or complication responsible for fatty liver, selected from the group consisting of obesity, visceral fat, metabolic syndrome and diabetes.

In another certain embodiment, the patient diagnosed with liver cirrhosis based on fatty liver disease by image findings or histology may satisfy the following criteria (4) to (6):
(4) an alcohol intake is more than 20 g and 70 g or less per day in terms of ethanol consumption;
(5) other known causes of liver damage have not been identified; and
(6) the patient has at least one possible condition or complication responsible for fatty liver, selected from the group consisting of obesity, visceral fat, metabolic syndrome and diabetes.

In at least one embodiment, the patient diagnosed with liver cirrhosis may further satisfy, in addition to the respective criteria, one or more of the following criteria (7) to (11) at the start of treatment:
(7) a total bilirubin value is 3.0 mg/dL or less;
(8) a platelet count is 5.0×10⁴/μL or more;
(9) prothrombin activity is 70% or more;
(10) a serum creatine level is 1.5 mg/dL or less; and
(11) a serum albumin level is 4.0 g/dL or less.

(Methods of Collection for an Adipose Tissue-Derived Cell Population Including ADRCs)

In some embodiments, adipose tissue is processed to obtain a refined, enriched, concentrated, isolated, or purified population of adipose-derived cells, e.g., a population of ADRCs, useful in the embodiments disclosed herein, using a cell processing unit, gradient sedimentation, filtration, or a combination of any one or more of these approaches. In general, adipose tissue is first removed from a subject (e.g., a mammal, a domestic animal, a rodent, a horse, a dog, cat, or human) then it is processed to obtain a cell population, e.g., a population of ADRCs. For allogeneic transplantation, an appropriate donor can be selected using methods known in the art, for example, methods used for selection of bone marrow donors. The volume of adipose tissue collected from the patient can vary from about 1 cc to about 2000 cc and in some embodiments up to about 3000 cc. The volume of tissue removed will vary from patient to patient and will depend on a number of factors including but not limited to: age, body habitus, coagulation profile, hemodynamic stability, severity of insufficiency or injury, co-morbidities, and physician preference.

The adipose tissue can be obtained by any method known to a person of ordinary skill in the art. For example, the adipose tissue may be removed from a subject by suction-assisted lipoplasty, ultrasound-assisted lipoplasty, or excisional lipectomy. In addition, the procedures may include a combination of such procedures, such as a combination of excisional lipectomy and suction-assisted lipoplasty. If the tissue or some fraction thereof is intended for re-implantation into a subject, the adipose tissue should be collected in a manner that preserves the viability of the cellular component and that minimizes the likelihood of contamination of the tissue with potentially infectious organisms, such as bacteria or viruses. Thus, the tissue extraction should be performed in a sterile or aseptic manner to minimize contamination. Suction-assisted lipoplasty may be desired to remove the adipose tissue from a patient as it provides a minimally invasive method of collecting tissue with minimal potential for stem cell damage that may be associated with other techniques, such as ultrasound-assisted lipoplasty.

Accordingly, adipose tissue provides a rich source of a population of cells that is easily enriched for adipose-derived regenerative cells. Collection of adipose tissue is also more patient-friendly and is associated with lower morbidity than collection of a similar volume of, for example, skin or a much larger volume of tonsil.

For suction-assisted lipoplastic procedures, adipose tissue is collected by insertion of a cannula into or near an adipose tissue depot present in the patient followed by aspiration of the adipose into a suction device. In some embodiments, a small cannula may be coupled to a syringe, and the adipose tissue may be aspirated using manual force. Using a syringe or other similar device may be desirable to harvest relatively moderate amounts of adipose tissue (e.g., from 0.1 mL to several hundred milliliters of adipose tissue). Procedures employing these relatively small devices require only local anesthesia. Larger volumes of adipose tissue (e.g., greater than several hundred milliliters) may require general anesthesia at the discretion of the donor and the person performing the collection procedure. When larger volumes of adipose tissue are to be removed, relatively larger cannulas and automated suction devices may be employed.

Excisional lipectomy procedures include, and are not limited to, procedures in which adipose tissue-containing tissues (e.g., skin) is removed as an incidental part of the procedure; that is, where the primary purpose of the surgery is the removal of tissue (e.g., skin in bariatric or cosmetic surgery) and in which adipose tissue is removed along with the tissue of primary interest. Subcutaneous adipose tissue may also be extracted by excisional lipectomy in which the adipose tissue is excised from the subcutaneous space without concomitant removal of skin.

In at least one embodiment, the ADRCs of the present disclosure may be collected from subcutaneous adipose tissue by a tumescent liposuction method which involves injecting a solution containing a large amount of physiological saline supplemented with a local anesthetic and a hemostat to the subcutaneous layer of a fat collection site, then inserting a liposuction tube thereto, and aspirating the subcutaneous fat in vacuum.

The amount of tissue collected can depend on a number of variables including, but not limited to, the body mass index of the donor, the availability of accessible adipose tissue harvest sites, concomitant and pre-existing medications and conditions (such as anticoagulant therapy), and the clinical purpose for which the tissue is being collected. Experience with transplant of hematopoietic stem cells (bone marrow or umbilical cord blood-derived stem cells used to regenerate the recipient's blood cell-forming capacity) shows that engraftment is cell dose-dependent with threshold effects (Smith, et al., 1995; Barker, et al., 2001, both incorporated herein by reference in their entirety). Thus, it is possible that the general principle that “more is better” will be applied within the limits set by other variables and that where feasible the harvest will collect as much tissue as possible.

The adipose tissue that is removed from a patient is then collected into a device (e.g., cell processing unit, centrifuge, or filtration unit) for further processing so as to remove collagen, adipocytes, blood, and saline, thereby obtaining a cell population including adipose derived cells, e.g., adipose-derived cells including regenerative cells. Preferably the population of adipose derived cells containing ADRCs is free from contaminating collagen, adipocytes, blood, and saline. The major contaminating cells in adipose tissue (adipocytes) have low density and are easily removed by flotation.

Adipose tissue processing to obtain a refined, concentrated, and isolated population of adipose-derived cells, e.g., a population of ADRCs, and modifications thereto are preferably performed using methods described, for example, in U.S. application Ser. No. 10/316,127 (U.S. patent application Pub. No. 2003/0161816), entitled SYSTEMS AND METHODS FOR TREATING PATIENTS WITH PROCESSED LIPOASPIRATE CELLS, filed on Dec. 9, 2002, and U.S. application Ser. No. 10/877,822 (U.S. patent application Pub. No. 2005/0084961), entitled SYSTEMS AND METHODS FOR SEPARATING AND CONCENTRATING REGENERATIVE CELLS FROM TISSUE, filed on Jun. 25, 2004; U.S. application Ser. No. 10/242,094, entitled PRESERVATION OF NON EMBRYONIC CELLS FROM NON HEMATOPOIETIC TISSUES, filed on Sep. 12, 2002, which claims the benefit of U.S. application. Ser. No. 60/322,070 filed on Sep. 14, 2001; U.S. application Ser. No. 10/884,638, entitled SYSTEMS AND METHODS FOR ISOLATING AND USING CLINICALLY SAFE ADIPOSE DERIVED REGENERATIVE CELLS, filed on Jul. 2, 2004; all of which are hereby expressly incorporated by reference in their entireties. The applications above disclose the processing of adipose-derived cells in a system that is configured to maintain a closed, sterile fluid/tissue pathway. This can be achieved by use of a pre-assembled, linked set of closed, sterile containers and tubing allowing for transfer of tissue and fluid elements within a closed pathway. This processing set can be linked to a series of processing reagents (e.g., saline, enzymes, etc.) inserted into a device, which can control the addition of reagents, temperature, and timing of processing thus relieving operators of the need to manually manage the process. In at least one embodiment, the entire procedure from tissue extraction through processing and placement into the recipient is performed in the same facility, indeed, even within the same room, of the patient undergoing the procedure.

For many applications, preparation of the active cell population requires depletion of the mature fat-laden adipocyte component of adipose tissue. This can be achieved by a series of washing and disaggregation steps in which the tissue is first rinsed to reduce the presence of free lipids (released from ruptured adipocytes) and peripheral blood elements (released from blood vessels severed during tissue harvest), and then disaggregated to free intact adipocytes and other cell populations from the connective tissue matrix. In some embodiments, the adipose-derived cells, e.g., ADRCs, are provided with blood vessel endothelial cells (BECs), BEC progenitors (EPCs), and adipose tissue-derived stem cells, adipose tissue-derived stromal cells, and other cellular elements. In some embodiments the adipose-derived cells, e.g., ADRCs, comprise cells that are in the form of aggregates or partially disaggregated fragments, for example, two or more vascular cells linked by extracellular matrix. In some embodiments such aggregates comprise large aggregates or fragments including more than 10 cells or more than 100 cells linked by extracellular matrix. Such aggregates may include but are not limited to blood or lymph vessel fragments in which several cells remain linked in an approximation of their original orientation to one another (including, by way of non-limiting example, vascular endothelial cells and pericytes or smooth muscle cells linked by some or all of the extracellular matrix that bound them together in the tissue prior to processing). In a particular embodiment such aggregates may comprise several hundred cells in contact or associated with fewer adipocytes than they were in the tissue prior to processing.

Rinsing is an optional but preferred step, wherein the tissue is mixed with a solution to wash away free lipid and single cell components, such as those components in blood, leaving behind intact adipose tissue fragments. In one embodiment, the adipose tissue that is removed from the patient is mixed with isotonic saline or other physiologic solution(s), e.g., Plasmalyte® of Baxter Inc. or Normosol® of Abbott Labs. Intact adipose tissue fragments can be separated from the free lipid and cells by any means known to persons of ordinary skill in the art including, but not limited to, filtration, decantation, sedimentation, or centrifugation. In some embodiments, the adipose tissue is separated from non-adipose tissue by employing a filter disposed within a tissue collection container, as discussed herein. In other embodiments, the adipose tissue is separated from non-adipose tissue using a tissue collection container that utilizes decantation, sedimentation, or centrifugation techniques to separate the materials.

The intact tissue fragments are then disaggregated using any conventional techniques or methods, including mechanical force (mincing or shear forces), ultrasonic or other physical energy, lasers, microwaves, enzymatic digestion with single or combinatorial proteolytic enzymes, such as collagenase, trypsin, lipase, liberase HI, nucleases, or members of the Blendzyme family as disclosed in U.S. Pat. No. 5,952,215, “Enzyme composition for tissue dissociation,” expressly incorporated herein by reference in its entirety, and pepsin, or a combination of mechanical and enzymatic methods. For example, the cellular component of the intact tissue fragments may be disaggregated by methods using collagenase-mediated dissociation of adipose tissue, similar to the methods for collecting microvascular endothelial cells in adipose tissue, as disclosed in U.S. Pat. No. 5,372,945, expressly incorporated herein by reference in its entirety. Additional methods using collagenase that may be used are disclosed in, e.g., U.S. Pat. No. 5,830,741. “Composition for tissue dissociation containing collagenase I and II from clostridium histolyticum and a neutral protease” and by Williams, et al., 1995, “Collagenase lot selection and purification for adipose tissue digestion,” Cell Transplant 4(3):281-9, both expressly incorporated herein by reference in their entirety. Similarly, a neutral protease may be used instead of collagenase, as disclosed in Twentyman, et al. (Twentyman, et al., 1980, “Use of bacterial neutral protease for disaggregation of mouse tumours and multicellular tumor spheroids,” Cancer Lett. 9(3):225-8, expressly incorporated herein by reference in its entirety). Furthermore, the methods described herein may employ a combination of enzymes, such as a combination of collagenase and trypsin or a combination of an enzyme, such as trypsin, and mechanical dissociation.

Adipose tissue-derived cells, e.g., ADRCs, may then be obtained from the disaggregated tissue fragments by reducing the number of mature adipocytes. A suspension of the disaggregated adipose tissue and the liquid in which the adipose tissue was disaggregated is then passed to another container, such as a cell collection container. The suspension may flow through one or more conduits to the cell collection container by using a pump, such as a peristaltic pump, that withdraws the suspension from the tissue collection container and urges it to the cell collection container. Other embodiments may employ the use of gravity or a vacuum while maintaining a closed system. Separation of the cells in the suspension may be achieved by buoyant density sedimentation, centrifugation, elutriation, filtration, differential adherence to and elution from solid phase moieties, antibody-mediated selection, differences in electrical charge, immuno-magnetic beads, fluorescence activated cell sorting (FACS), or other means. Examples of these various techniques and devices for performing the techniques may be found in U.S. Pat. Nos. 6,277,060; 6,221,315; 6,043,066; 6,451,207; 5,641,622; and 6,251,295, all incorporated herein by reference in their entirety. Many of these devices can be incorporated within the cell processing unit, while maintaining a closed system.

In some embodiments, the cells in the suspension are separated from the acellular component of the suspension using a spinning membrane filter. In other embodiments, the cells in the suspension are separated from the acellular component using a centrifuge. In one such exemplary embodiment the cell collection container may be a flexible bag that is structured to be placed in a centrifuge (e.g., manually or by robotics). In other embodiments, a flexible bag is not used. After centrifugation, the cellular component containing ADRCs forms a pellet, which may then be re-suspended with a buffered solution so that the cells can be passed through one or more conduits to a mixing container, as discussed herein. The resuspension fluids may be provided by any suitable means. For example, a buffer may be injected into a port on the cell collection container, or the cell collection container may include a reserve of buffer that can be mixed with the pellet of cells by rupturing the reserve. When a spinning membrane filter is used, resuspension is optional since the cells remain in a volume of liquid after the separation procedure.

In one embodiment a subpopulation of the adipose-derived cells, e.g., ADRCs, is selected from other cells by short term adherence to a surface, for example, plastic. In one embodiment the duration of adherence for the purpose of selection is approximately one hour. In a second embodiment the duration of adherence to the surface is 24 hours.

Although some embodiments described herein are directed to methods of fully disaggregating the adipose tissue to separate the active cells from the mature adipocytes and connective tissue, additional embodiments are directed to methods in which the adipose tissue is only partially disaggregated. For example, partial disaggregation may be performed with one or more enzymes, which are removed from at least a part of the adipose tissue early relative to an amount of time that the enzyme would otherwise be left thereon to fully disaggregate the tissue. Such a process may require less processing time and would generate fragments of tissue components within which multiple adipose-derived cells, e.g., ADRCs remain in partial or full contact. In another embodiment mechanical force (for example ultrasound energy or shear force) is applied to prepare the cells, e.g., ADRCs, or fragments including adipose-derived cells isolated from all or some of the mature adipocytes with which they were associated in the tissue prior to processing.

In some embodiments, the tissue is washed with sterile buffered isotonic saline and incubated with collagenase at a collagenase concentration, a temperature, and for a period of time sufficient to provide adequate disaggregation. In at least one embodiment, the collagenase enzyme used will be approved for human use by the relevant authority (e.g., the U. S. Food and Drug Administration). Suitable collagenase preparations include recombinant and non-recombinant collagenase. Non-recombinant collagenase may be obtained from F. Hoffmann-La Roche Ltd., Indianapolis, IN or Advance Biofactures Corp., Lynbrook, NY. Recombinant collagenase may also be obtained as disclosed in U.S. Pat. No. 6,475,764.

In one embodiment, solutions contain collagenase at concentrations of about 10 μg/mL to about 50 μg/mL (e.g., 10 μg/mL, 20 μg/mL, 30 μg/mL, 40 μg/mL, or 50 μg/mL) and are incubated at from about 30° C. to about 38° C. for from about 20 minutes to about 60 minutes. These parameters will vary according to the source of the collagenase enzyme, optimized by empirical studies, in order to confirm that the system is effective at extracting the desired cell populations in an appropriate time frame. A particular preferred concentration, time and temperature is 20 μg/mL collagenase (mixed with the neutral protease dispase; Blendzyme 1, Roche) and incubated for 45 minutes at about 37° C. In another embodiment. 0.5 units/mL collagenase (mixed with the neutral protease thermolysin; Blendzyme 3) is used In another embodiment, the collagenase enzyme used is material approved for human use by the relevant authority (e.g., the U.S. Food and Drug Administration). The collagenase used should be free of micro-organisms and contaminants, such as endotoxin.

Following disaggregation, the active cell population can be washed/rinsed to remove additives or by-products of the disaggregation process (e.g., collagenase and newly-released free lipid). The active cell population can then be concentrated by centrifugation or other methods known to persons of ordinary skill in the art, as discussed above. These post-processing wash/concentration steps may be applied separately or simultaneously. In one embodiment, the adipose-derived cells, e.g., ADRCs, are concentrated and the collagenase removed by passing the cell population through a continuous flow spinning membrane system or the like, such as, for example, the system disclosed in U.S. Pat. Nos. 5,034.135 and 5,234,608, all incorporated herein by reference in their entirety.

In addition to the foregoing, there are many known post-wash methods that may be applied for further purifying the adipose-derived cell population that comprises ADRCs. These include both positive selection (selecting the target cells), negative selection (selective removal of unwanted cells), or combinations thereof. In addition to separation by flow cytometry as described herein and in the literature, cells can be separated based on a number of different parameters, including, but not limited to, charge or size (e.g., by dielectrophoresis or various centrifugation methods, etc.).

Many other conformations of the staged mechanisms used for cell processing will be apparent to one skilled in the art. For example, mixing of tissue and saline during washing and disaggregation can occur by agitation or by fluid recirculation. Cell washing may be mediated by a continuous flow mechanism such as the spinning membrane approach, differential adherence, differential centrifugation (including, but not limited to differential sedimentation, velocity, or gradient separation), or by a combination of means. Similarly, additional components allow further manipulation of cells, including addition of growth factors or other biological response modifiers, and mixing of cells with natural or synthetic components intended for implant with the cells into the recipient.

Post-processing manipulation may also include cell culture or further cell purification (Kriehuber, et al., 2001; Garrafa, et al., 2006). In some embodiments, once the adipose-derived cell population, cells, e.g., ADRCs, is obtained, it is further refined, concentrated, enriched, isolated, or purified using a cell sorting device or gradient sedimentation. Mechanisms for performing these functions may be integrated within the described devices or may be incorporated in separate devices. In many embodiments, however, a therapeutically effective amount of a concentrated population of adipose derived cells, e.g., adipose-derived cells including regenerative cells, is used to prepare a medicament for prevention or treatment of liver fibrosis or liver cirrhosis, wherein said concentrated population of cells is to be administered to a patient in need thereof without culturing the cells before administering them to the patient. That is, some embodiments concern methods to prevention or treatment of liver fibrosis or liver cirrhosis, wherein a therapeutically effective amount of a concentrated population of adipose derived cells. e.g., ADRCs, is administered to a patient in need thereof without culturing the cells before administering them to the patient.

In at least one embodiment, the tissue removal system and processing set would be present in the vicinity of the patient receiving the treatment, such as the operating room or out-patient procedure room (effectively at the patient's bedside). This allows rapid, efficient tissue harvest and processing, and decreases the opportunity for specimen handling/labeling error, thereby allowing for performance of the entire process in the course of a single surgical procedure.

As described in U.S. application Ser. No. 10/884,638, entitled SYSTEMS AND METHODS FOR ISOLATING AND USING CLINICALLY SAFE ADIPOSE DERIVED REGENERATIVE CELLS, filed on Jul. 2, 2004, one or more additives may be added to the cells during or after processing. Some examples of additives include agents that optimize washing and disaggregation, additives that enhance the viability of the active cell population (e.g., adipose-derived cells including regenerative cells), during processing, anti-microbial agents (e.g., antibiotics), additives that lyse adipocytes or red blood cells, or additives that enrich for cell populations of interest (by differential adherence to solid phase moieties or to otherwise promote the substantial reduction or enrichment of cell populations).

The adipose-derived cells, e.g., ADRCs, obtained as described herein can be cultured according to approaches known in the art, and the cultured cells can be used in several of the embodied methods. For example, adipose-derived cells, e.g., ADRCs including regenerative cells, can be cultured on collagen-coated dishes or 3D collagen gel cultures in endothelial cell basal medium in the presence of low or high fetal bovine serum or similar product, as described in Ng, et al., November 2004, “Interstitial flow differentially stimulates blood and lymphatic endothelial cell morphogenesis in vitro,” Microvasc Res. 68(3):258-64, incorporated herein by reference. Alternatively, adipose-derived cells, e.g., ADRCs, can be cultured on other extracellular matrix protein-coated dishes. Examples of extracellular matrix proteins that may be used include, but are not limited to, fibronectin, laminin, vitronectin, and collagen IV. Gelatin or any other compound or support, which similarly promotes adhesion of endothelial cells into culture vessels may be used to culture ADRCs, as well.

Examples of basal culture medium that can be used to culture adipose-derived cells, e.g., ADRCs, in vitro include, but are not limited to, EGM, RPMI, M199, MCDB131, DMEM, EMEM, McCoy's 5A, Iscove's medium, modified Iscove's medium or any other medium known in the art to support the growth of blood endothelial cells. Examples of supplemental factors or compounds that can be added to the basal culture medium that could be used to culture ADRCs include, but are not limited to, ascorbic acid, heparin, endothelial cell growth factor, endothelial growth supplement, glutamine, HEPES, Nu serum, fetal bovine serum, human serum, equine serum, plasma-derived horse serum, iron-supplemented calf serum, penicillin, streptomycin, amphotericin B, basic and acidic fibroblast growth factors, insulin-growth factor, astrocyte conditioned medium, fibroblast or fibroblast-like cell conditioned medium, sodium hydrogencarbonate, epidermal growth factor, bovine pituitary extract, magnesium sulphate, isobutylmethylxanthine, hydrocortisone, dexamethasone, dibutyryl cyclic AMP, insulin, transferrin, sodium selenite, oestradiol, progesterone, growth hormone, angiogenin, angiopoietin-1, Del-1, follistatin, granulocyte colony-stimulating factor (G-CSF), erythropoietin, hepatocyte growth factor (HGF)/scatter factor (SF), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular endothelial growth factor (VEGF)/vascular permeability factor (VPF), interleukin-3 (IL-3), interleukin 7 (IL-7), interleukin-8 (IL-8), ephrins, matrix metalloproteinases (such as MMP2 and MMP9), or any other compound known in the art to promote survival, proliferation or differentiation of endothelial cells.

Further processing of the cells may also include: cell expansion (of one or more regenerative cell types) and cell maintenance (including cell sheet rinsing and media changing); sub-culturing; cell seeding; transient transfection (including seeding of transfected cells from bulk supply); harvesting (including enzymatic, non-enzymatic harvesting and harvesting by mechanical scraping); measuring cell viability; cell plating (e.g., on microtiter plates, including picking cells from individual wells for expansion, expansion of cells into fresh wells): high throughput screening; cell therapy applications; gene therapy applications; tissue engineering applications; therapeutic protein applications; viral vaccine applications; harvest of regenerative cells or supernatant for banking or screening, measurement of cell growth, lysis, inoculation, infection or induction: generation of cell lines (including hybridoma cells); culture of cells for permeability studies; cells for RNAi and viral resistance studies; cells for knock-out and transgenic animal studies; affinity purification studies; structural biology applications; assay development and protein engineering applications.

In general, a system useful for isolating a population of adipose tissue-derived cells, e.g., a population of ADRCs, comprises a) a tissue collection container including i) a tissue collecting inlet port structured to receive adipose tissue removed from a subject, and ii) a filter disposed within the tissue collection container, which is configured to retain the adipose-derived cell population from said subject and to pass adipocytes, blood, and saline; b) a mixing container or cell processing chamber coupled to the tissue collection container by a conduit such that a closed pathway is maintained, wherein said mixing container receives said cell population and said mixing container comprises an additive port for introducing at least one additive to said population of adipose-derived cells; and an outlet port configured to allow removal of said population of adipose-derived cells from the mixing container or cell processing chamber for administration to a patient. In some embodiments, said mixing container or cell processing container further comprises a cell concentration device such as a spinning membrane filter or a centrifuge. Aspects of the embodiments disclosed herein also include a cell sorter, which is attached to said mixing chamber or cell processing chamber by a conduit and is configured to receive cells from said mixing chamber or cell processing chamber, while maintaining a closed pathway. Aspects of the embodiments above may also include a centrifuge attached to said mixing chamber or cell processing chamber by a conduit and configured to receive said population of adipose derived cells, while maintaining a closed pathway, wherein said centrifuge comprises a gradient suitable for further separation and purification of said population of adipose-derived cells (e.g., ficoll-hypaque). Said centrifuge containing said gradient, which is configured to receive said population of adipose-derived cells may also be contained within said mixing container or cell processing chamber.

In at least one embodiment, isolation of the ADRCs of the present disclosure can be performed by washing the collected adipose tissue, and enzymatically or mechanically disaggregating the tissue to release cells bound in the adipose tissue matrix. For example, methods or apparatuses described in U.S. Pat. Nos. 7,390,484, 7,585,670, 7,687,059, 8,309,342, and 8,440,440, U.S. Patent Application Publication Nos. 2013/0164731 and 2008/0014181, and International Publication Nos. WO2009/073724 and WO2013/030761 may be used for the isolation of the ADRCs. A completely aseptic closed-type adipose tissue separation apparatus (Celution® 800/IV; Cytori Therapeutics Inc.) may be used for the isolation of the ADRCs of the present disclosure.

(Characterization of Adipose Tissue-Derived Cell Population Including ADRCs)

A measurement, analysis, or characterization of the population of adipose-derived cells described herein to determine the presence of certain cells in the population can be undertaken within the closed system of a cell processing unit or outside of the closed system of a cell processing unit using any number of protein or RNA detection assays available in the art. Additionally, the measurement, analysis, or characterization of the adipose-derived cells, or certain cells included in ADRCs (e.g., stem cells, progenitor cells, precursor cells, and the like), can be part of or can accompany the isolation procedure (e.g., cell sorting using an antibody specific for certain cell types (e.g., regenerative cells) or gradient separation using a media selective for certain cell types).

In some embodiments the measurement or characterization of the isolated cell population is conducted by detecting the presence or absence of a protein marker that is unique to certain cell types (e.g., adipose-derived regenerative cells, adipose-derived stem cells, or the like) is otherwise considered to confirm the presence of the specific cell type of interest by those of skill in the art. In addition to conventional Western blots using antibody probes specific for said proteins or markers, immuno-selection techniques that exploit on cell surface marker expression can be performed using a number of methods known in the art and described in the literature. Such approaches can be performed using an antibody that is linked directly or indirectly to a solid substrate (e.g., magnetic beads) in conjunction with a manual, automated, or semi-automated device as described by Watts, et al., for separation of CD34-positive cells (Watts, et al., 2002, “Variable product purity and functional capacity after CD34 selection: a direct comparison of the CliniMACS (v2.1) and Isolex 300i (v2.5) clinical scale devices,” Br J Haematol. 2002 July; 118(1):117-23), by panning, use of a Fluorescence Activated Cell Sorter (FACS), or other means.

Separation, measurement, and characterization can also be achieved by positive selection using antibodies that recognize cell surface markers or marker combinations that are expressed by certain cell types, but not by one or more of the other cell types or sub-populations present within the cell population. Separation, measurement, and characterization can also be achieved by negative selection, in which non-desired cell types are removed from the isolated population of adipose-derived cells using antibodies or antibody combinations that do not exhibit appreciable binding to ADRCs. Markers that are specifically expressed by ADRCs have been described. Examples of antibodies that could be used in negative selection include, but are not limited to, markers expressed by endothelial cells. There are many other antibodies well known in the art that can be applied to negative selection. The relative specificity of markers for ADRCs can also be exploited in a purification or characterization or measurement strategy. For example, a fluorescently-labeled ligand can be used in FACS-based sorting of cells, or a ligand conjugated directly or indirectly to a solid substrate can be used to separate in a manner analogous to the immuno-selection approaches described above.

Measurement and characterization of the adipose-derived cell population to determine the presence or absence of specific cell types (e.g., specific types of regenerative cells) can also involve analysis of one or more RNAs that encode a protein that is unique to or otherwise considered by those of skill in the art to be a marker that indicates the presence or absence of a ADRCs. In some embodiments, for example, the isolated cell population or a portion thereof is analyzed for the presence or absence of an RNA that encodes one or more of, e.g., CD45. The detection of said RNAs can be accomplished by any techniques available to one of skill in the art, including but not limited to, Northern hybridization, PCR-based methodologies, transcription run-off assays, gene arrays, and gene chips.

In at least one embodiment, the ADRCs of the present disclosure can express CD31⁻. In at least one embodiment, the ADRCs also can express CD34⁺, e.g., the ADRCs of the present disclosure is CD31⁻ and CD34⁺. In at least one embodiment, the ADRCs are CD45⁻. In other word, the ADRCs are CD31⁻. CD34⁺ and CD45⁻. In another embodiment, the ADRCs are CD146⁺. In some embodiments, the cells are CD31⁻, CD34⁺, CD45⁻, and CD146⁺.

In another embodiment the ADRCs comprise cells that are CD45⁺ and in at least one embodiment is CD45⁺ and CD206⁺.

In some embodiments, the ADRCs express an amount of, e.g., CD45, CD11b, CD14, CD68, CD90, CD73, CD31 or CD34.

In some approaches, ADRCs are CD14⁺ or CD11b⁺.

In at least one embodiment, the ADRCs of the present disclosure may be identified by expression of cell surface marker selected from the group consisting of CD34, CD44, CD45, CD90 and CD105. In some approaches. ADRCs are CD34⁺, CD44⁺, CD45⁺, CD90⁺ and CD105⁺.

(Method for Preparation of Pharmaceutical Compositions Including ADRCs)

In accordance with the aforementioned approaches, raw adipose tissue is processed to substantially remove mature adipocytes and connective tissue thereby obtaining a heterogeneous plurality of adipose tissue-derived cells including adipose-derived cells, e.g., ADRCs, suitable for placement within the body of a subject. The extracted adipose-derived cells, e.g., ADRCs, may be provided in a neat composition including these cells substantially free from mature adipocytes and connective tissue or in combination with an inactive ingredient (e.g., a carrier) or a second active ingredient (e.g., adipose-derived stem cell or adipose-derived endothelial cell). The cells may be placed into the recipient alone or in combination (e.g., in a single composition or co-administered) with biological materials, such as cells, tissue, tissue fragments, or stimulators of cell growth or differentiation, supports, prosthetics, or medical devices. The composition may include additional components, such as cell differentiation factors, growth promoters, immunosuppressive agents, or medical devices, as discussed herein, for example. In some embodiments, the cells, with any of the above mentioned additives, are placed into the person from whom they were obtained (e.g., autologous transfer) in the context of a single operative procedure with the intention of providing a therapeutic benefit to the recipient.

Accordingly, aspects of the invention include compositions that comprise, consist, or consist essentially of a refined, enriched, concentrated, isolated, or purified adipose-derived cell population, e.g., ADRCs, with a biological material, additive, support, prosthetic, or medical device, including but not limited to, unprocessed adipose tissue, adipocytes, collagen matrix or support, cell differentiation factors, growth promoters, immunosuppressive agents, processed adipose tissue containing adipose-derived stem cells or progenitor cells, and cell populations already containing an enriched amount of ADRCs. In some embodiments, the aforementioned compositions comprise an amount or concentration of refined, isolated, or purified ADRCs that is greater than or equal to 0.5%-1%, 1%-2%, 2%-4%, 4%-6%, 6%-8%, 8%-10%, 10%-20%, 20%-30%, 30%-40%, 40%-50%, 50%-60%, 60%-70%, 70%-80%, 80%-90%, or 90%-100% ADRCs, as compared to the total adipose-tissue cell population.

In some embodiments, the adipose-derived cell e.g., ADRCs, described herein is formulated in compositions that include at least one pharmaceutically acceptable diluent, adjuvant, or carrier substance, using any available pharmaceutical chemistry techniques. Generally, this entails preparing compositions that are essentially free of impurities that could be harmful to humans or animals.

Appropriate salts and buffers can be employed to stabilize and to facilitate uptake of the adipose-derived cell population that comprises ADRCs. Compositions contemplated herein can comprise an effective amount of the adipose-derived cells e.g., ADRCs in a pharmaceutically acceptable carrier or aqueous medium.

Administration of the compositions described herein can be via any common route so long as the target tissue is available via that route. Compositions administered according to the methods described herein may be introduced into the subject by, e.g., by intravenous, intraarterial, intralymphatic, subcutaneous, intradermal, intramuscular, intramammary, intraperitoneal, intrathecal, retrobulbar, intrapulmonary (e.g., term release); by oral, sublingual, nasal, anal, vaginal, or transdermal delivery, by spray or other direct application, or by surgical implantation at a particular site. In at least one embodiment, the pharmaceutical composition of the present disclosure including ADRCs are administered by the form of, for example, intra-arterial, intravenous, intraportal, intradermal, subcutaneous, intramuscular or intraperitoneal injection, though the administration method is not limited thereto.

Administration by intra-arterial, intravenous or intraportal injection is preferred, and hepatic artery administration is particularly preferably performed, because administered ADRCs reach the liver, an organ to be treated, through blood circulation.

In the specific embodiment, a single cell suspension of ADRCs is prepared for intra-arterial, intravenous or intraportal injection.

Intravenous and intraarterial cell transplantations are associated with a number of risk and complications. Cell size and diameter are major determinants of vascular obstruction and complications emerging thereof. Pulmonary microembolism is an important safety concern which has been reported after both intravenous and intraarterial cell delivery, particularly when injecting larger cells.

Adipose tissue processing may lead to cell rupture and release of extracellular debris and DNA that result in cell aggregation or clumping. The sticky nature of DNA causes cells and other debris to aggregate into large clumps. A DNase I enzyme can be used to digest free/loose DNA strands. Alternatively, a highly purified DNase I enzyme can be used for efficient, gentle and reproducible digestion of free/loose DNA, leading to the disaggregation of ADRCs and prevents such cells from sticking to each other, thus producing a single cell suspension of ADRCs. Such an enzyme is commercially available as Intravase® 840 from Cytori Therapeutics Inc.

Moreover, the removal of cellular aggregates and debris through the use of a filter membrane screen immediately prior to intravenous or intraarterial cell delivery into the patient mitigates the risk of vascular obstruction and consequent complications. Any filter membrane screen with an appropriate pore size can be used for the above purpose. In at least one embodiment, the filter membrane screen is designed to mate with the delivery mechanism contacting the patient, and filter the single cell suspension of ADRCs being infused into the patient. Such a filter is intended to allow single and diploidal cells to pass through the membrane and onto the patient while trapping and preventing the passage of large cellular aggregates that could cause or contribute to the formation of microemboli in the microvasculature of the patient.

For example, the Macro Syringe Filter, which is a sterile, non-pyrogenic, single use, disposable syringe filter composed of an acrylic housing that encases a polyester filter membrane screen with an average pore size of 43 microns, is commercially available from Cytori Therapeutics, Inc.

In each of these methods of administration the compositions may or may not comprise a carrier or other material that has the property of increasing retention of the composition at the site of action or of facilitating the traffic of the composition to the site of action. The introduction may consist of a single dose or a plurality of doses over a period of time. Vehicles for cell therapy agents are known in the art and have been described in the literature. See, for example Remington's Pharmaceutical Sciences, 18th Ed. (1990, Mack Publ. Co, Easton Pa. 18042) pp 1435-1712, incorporated herein by reference. Sterile solutions are prepared by incorporating the adipose-derived cell population e.g., adipose-derived cells including stem cells, adipose-derived cells including regenerative cells, adipose-derived cells including stem and regenerative cells, and the like, in the required amount in the appropriate buffer with or without one or more of the other components described herein.

Combination therapy with any two or more agents described herein also is contemplated as an aspect of the invention. Similarly, every combination of agents described herein, packaged together as a new kit, or formulated together as a single composition, is considered an aspect of the invention. Compositions for use according to aspects of the invention preferably include the adipose-derived cell population e.g., ADRCs, formulated with a pharmaceutically acceptable carrier. The cells can also be applied with additives to enhance, control, or otherwise direct the intended therapeutic effect. For example, in some embodiments, the adipose-derived cell population e.g., ADRCs, are further purified by use of antibody-mediated positive or negative cell selection to enrich the cell population to increase efficacy, reduce morbidity, or to facilitate ease of the procedure. Similarly, cells can be applied with a biocompatible matrix, which facilitates in vivo tissue engineering by supporting or directing the fate of the implanted cells. In the same way, cells can be administered following genetic manipulation such that they express gene products that are believed to or are intended to promote the therapeutic response provided by the cells.

The adipose-derived cell population, e.g., ADRCs, can be applied alone or in combination with other cells, tissue, tissue fragments, growth factors, biologically active or inert compounds, resorbable plastic scaffolds, or other additive intended to enhance the delivery, efficacy, tolerability, or function of the population. The adipose-derived cell population that comprises ADRCs can also be modified by insertion of DNA or by placement in cell culture in such a way as to change, enhance, or supplement the function of the cells for derivation of a structural or therapeutic purpose.

In some embodiments, the adipose-derived cell population e.g., ADRCs, are combined with a gene encoding a pro-drug converting enzyme which allows cells to activate pro-drugs within the site of engraftment, that is, within a tumor. Addition of the gene (or combination of genes) can be by any technology known in the art including but not limited to adenoviral transduction, “gene guns,” liposome-mediated transduction, and retrovirus or lentivirus-mediated transduction, plasmid, or adeno-associated virus. Cells can be implanted along with a carrier material bearing gene delivery vehicle capable of releasing or presenting genes to the cells over time such that transduction can continue or be initiated in situ. Particularly when the cells or tissue containing the cells are administered to a patient other than the patient from whom the cells or tissue were obtained, one or more immunosuppressive agents can be administered to the patient receiving the cells or tissue to reduce, and preferably prevent, rejection of the transplant.

Some embodiments concern the ex vivo transfection of an adipose-derived cell population, e.g., ADRCs, and subsequent transfer of these transfected cells to subjects. It is contemplated that such embodiments can be an effective approach to upregulate in vivo levels of the transferred gene and for providing relief from a disease or disorder resulting from under-expression of the gene(s) or otherwise responsive to upregulation of the gene (see e.g., Gelse, et al., 2003, “Articular cartilage repair by gene therapy using growth factor-producing mesenchymal cells,” Arthritis Rheum. 48:430-41; Huard, et al, 2002, “Muscle-derived cell-mediated ex vivo gene therapy for urological dysfunction,” Gene Ther. 9:1617-26; Kim, et al., 2002. “Ex vivo gene delivery of IL-1 Ra and soluble TNF receptor confers a distal synergistic therapeutic effect in antigen-induced arthritis,” Mol. Ther. 6:591-600, all incorporated herein by reference). Delivery of an adipose-derived cell population, e.g., ADRCs, to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, 1998, “Human Gene Therapy,” Nature Suppl. to vol. 392 (6679):25-20, incorporated by reference herein. Gene therapy technologies are also reviewed by Friedmann, 1989, “Progress toward human gene therapy,” Science 244(4910): 1275-1281, Verma (1990), “Gene therapy.” Scientific American 263(5): 68-84, and Miller (1992), “Human gene therapy comes of age,” Nature, 357:455-460, all incorporated by reference herein. An adipose-derived cell population, e.g., ADRCs, can be cultured ex vivo in the presence of an additive (e.g., a compound that induces differentiation or pancreatic cell formation) in order to proliferate or to produce a desired effect on or activity in such cells. Treated cells can then be introduced to a subject.

Aspects of the invention also concern the ex vivo transfection of adipose-derived cells, e.g., ADRCs (stem cells, progenitor cells, precursor cells, or combinations of stem cells and progenitor cells or precursor cells) with a gene encoding a therapeutic polypeptide, and administration of the transfected cells to the mammalian subject.

In some embodiments, the administering step comprises implanting a prosthetic or medical device (e.g., intravascular stent) in the mammalian subject, where the stent is coated or impregnated with an adipose-derived cell population that comprises ADRCs. Exemplary materials for constructing valves, stents or grafts coated or seeded with transfected endothelial cells are described in Pavcnik, et al., 2004, “Second-generation percutaneous bioprosthetic valve: a short-term study in sheep,” Eur. J. Endovasc. Surg. 40:1223-1227, and Arts, et al., 2002, “Contaminants from the Transplant Contribute to Intimal Hyperplasia Associated with Microvascular Endothelial Cell Seeding,” Eur. J. Endovasc. Surg. 23:29-38, incorporated herein by reference. See also U.S. patent application Ser. No. 11/317,422, entitled CELL-LOADED PROSTHESIS FOR REGENERATIVE INTRALUMINAL APPLICATIONS, filed on Dec. 22, 2005, incorporated herein by reference. For example, in one variation, a synthetic valve that comprises an adipose-derived cell population that comprises ADRCs is sutured to a square stainless steel stent. The square stent has a short barb at each end to provide anchors for the valve during placement, and the submucosa membrane is slit at the diagonal axis of the stent to create the valve opening.

Surfaces of the synthetic valve can be coated with a transfected or non-transfected adipose-derived cell population that comprises ADRCs (e.g., that comprises stem cells, that comprises progenitor cells, that comprises precursor cells, or other regenerative cells—including any combination thereof) e.g., by placing the synthetic valve in an appropriate cell culture medium for 1-3 days prior to implantation to allow for complete coverage of valve surface with the cells.

In another embodiment, the administering step comprises implanting an intravascular stent in the mammalian subject, where the stent is coated or impregnated, as described in literature cited above and reviewed in Lincoff, et al., 1994. A metal or polymeric wire for forming a stent is coated with a composition such as a porous biocompatible polymer or gel that is impregnated with (or can be dipped in or otherwise easily coated immediately prior to use with) a transfected or non-transfected adipose-derived cell population that comprises ADRCs (e.g., that comprises stem cells, that comprises progenitor cells, that comprises precursor cells, or other regenerative cells—including any combination thereof). The wire is coiled, woven, or otherwise formed into a stent suitable for implantation into the lumen of a vessel using conventional materials and techniques, such as intravascular angioplasty catheterization. Exemplary stents that may be improved in this manner are described and depicted in U.S. Pat. Nos. 5,800,507 and 5,697,967 (Medtronic, Inc., describing an intraluminal stent including fibrin and an elutable drug capable of providing a treatment of restenosis); U.S. Pat. No. 5,776,184 (Medtronic, Inc., describing a stent with a porous coating including a polymer and a therapeutic substance in a solid or solid/solution with the polymer); U.S. Pat. No. 5,799,384 (Medtronic, Inc., describing a flexible, cylindrical, metal stent having a biocompatible polymeric surface to contact a body lumen); and U.S. Pat. Nos. 5,824,048, 5,679,400 and 5,779,729; all of which are hereby expressly incorporated herein by reference in their entirety.

As disclosed herein, the adipose-derived cell population that comprises ADRCs (e.g., that comprises stem cells, that comprises progenitor cells, that comprises precursor cells, or other regenerative cells—including any combination thereof) may be provided to the subject, without further processing or following additional procedures to further purify, modify, stimulate, or otherwise change the cells. For example, the cells obtained from a patient may be provided back to said patient without culturing the cells before administration. In several embodiments, the collection and processing of adipose tissue, as well as, administration of the adipose-derived cell population that comprises ADRCs is performed at a patient's bedside. In a preferred embodiment, the cells are extracted from the adipose tissue of the person into whom they are to be implanted, thereby reducing potential complications associated with antigenic or immunogenic responses to the transplant. However, the use of cells extracted from another individual is also contemplated.

In at least one embodiment, the adipose tissue-derived cells are delivered to the patient soon after harvesting the adipose tissue from the patient. For example, the cells may be administered immediately after the processing of the adipose tissue to obtain a composition of ADRCs. The timing of delivery will depend upon patient availability and the time required to process the adipose tissue. In another embodiment, the timing for delivery may be relatively longer if the cells to be delivered to the patient are subject to additional modification, purification, stimulation, or other manipulation, as discussed herein. Furthermore, the adipose-derived cell population that comprises ADRCs may be administered multiple times. For example, the cells may be administered continuously over an extended period of time (e.g., hours), or may be administered in multiple injections extended over a period of time.

The number of the adipose-derived cells, e.g., ADRCs administered to a patient may be related to the cell yield after adipose tissue processing. In addition, the dose delivered will depend on the route of delivery of the cells to the patient. The cell dose administered to the patient will also be dependent on the amount of adipose tissue harvested and the body mass index of the donor (as a measure of the amount of available adipose tissue). The amount of tissue harvested will also be determined by the extent of the injury or insufficiency. Multiple treatments using multiple tissue harvests or using a single harvest with appropriate storage of cells between applications are within the scope of this invention.

A portion of the total number of adipose-derived cells, e.g., adipose-derived cells including regenerative cells, may be retained for later use or cryopreserved. Portions of the processed adipose tissue may be stored before being administered to a patient. For short term storage (e.g., less than 6 hours) cells may be stored at or below room temperature in a sealed container with or without supplementation with a nutrient solution. Medium term storage (e.g., less than 48 hours) is preferably performed at 2-8° C. in an isosmotic, buffered solution (for example Plasmalyte®) in a container composed of or coated with a material that prevents cell adhesion. Longer term storage is preferably performed by appropriate cryopreservation and storage of cells under conditions that promote retention of cellular function, such as disclosed in PCT App. No. PCT/US02/29207, filed on Sep. 13, 2002 and U.S. patent application Ser. No. 60/322,070, filed on Sep. 14, 2001, the contents of both of which are hereby expressly incorporated by reference.

In some embodiments, the amount of adipose derived cells (e.g., an enriched, concentrated, isolated, or purified population of the adipose-derived cells including ADRCs), which is provided to a subject in need thereof is greater than or equal to about 10,000, 20,000, 30,000, 40,000, 50,000, 60,000, 70,000, 80,000, 90,000, 100,000, 110,000, 120,000, 130,000, 140,000, 150,000, 160,000, 170,000, 180,000, 190,000, or 200,000 cells and the amount of ADRCs (e.g., the amount of stem cells, progenitor cells, precursor cells, or a combination of different types of regenerative cells—such as a combination of stem cells and progenitor cells) in said population of adipose derived cells can be greater than or equal to 0.5%-1%, 1%-2%, 2%-4%, 4%-6%, 6%-8%, 8%-10%, 10%-20%, 20%-30%, 30%-40%, 40%-50%, 50%-60%, 60%-70%, 70%-80%, 80%-90%, or 90%-100% of the total population of adipose derived cells. The percentage is based on total number of nucleated cells found in the adipose-derived cell population. The dose can be divided into several smaller doses, e.g., for administering over a period of time or for injection into different parts of the affected tissue, e.g., by local injection. However, this dosage can be adjusted by orders of magnitude to achieve the desired therapeutic effect.

The adipose-derived cell population, e.g., ADRCs, can be applied by several routes including systemic administration by venous or arterial infusion (including retrograde flow infusion and including infusion into blood or lymphatic vessels) or by direct injection. Systemic administration, particularly by peripheral venous access, has the advantage of being minimally invasive relying on the natural transport of cells from the blood to the liver. The adipose-derived cell population that comprises ADRCs can be injected in a single bolus, through a slow infusion, or through a staggered series of applications separated by several hours or, provided cells are appropriately stored, several days or weeks. The adipose-derived cell population that comprises ADRCs can also be applied by use of catheterization such that the first pass of cells through the area of interest is enhanced by using balloons. As with peripheral venous access, the adipose-derived cell population that comprises ADRCs may be injected through the catheters in a single bolus or in multiple smaller aliquots.

As previously set forth above, in a preferred embodiment, the adipose-derived cell population, e.g., ADRCs, is administered directly into the patient. In other words, the active cell population, e.g., the ADRCs, are administered to the patient without being removed from the system or exposed to the external environment of the system before being administered to the patient. Providing a closed system reduces the possibility of contamination of the material being administered to the patient. Thus, processing the adipose tissue in a closed system provides advantages over existing methods because the active cell population is more likely to be sterile. In some embodiments, the only time the adipose-derived cell population that comprises ADRCs are exposed to the external environment, or removed from the system, is when the cells are being withdrawn into an application device and administered to the patient. In other embodiments, the application device can also be part of the closed system. Accordingly, a complete closed system is maintained from removal of the adipose tissue from the subject (e.g., cannula) to introduction to the subject (e.g., application device). Thus, the cells used in these embodiments are may be processed for culturing or cryopreservation and may be administered to a patient without further processing, or may be administered to a patient after being mixed with other tissues, cells, or additives.

In other embodiments, at least a portion of the adipose-derived cell population that comprises ADRCs can be stored for later implantation/infusion. The population may be divided into more than one aliquot or unit such that part of the population of cells is retained for later application while part is applied immediately to the patient. Moderate to long-term storage of all or part of the cells in a cell bank is also within the scope of this invention, as disclosed in U.S. patent application Ser. No. 10/242,094, entitled PRESERVATION OF NON EMBRYONIC CELLS FROM NON HEMATOPOIETIC TISSUES, filed on Sep. 12, 2002, which claims the benefit of U.S. application. Ser. No. 60/322,070, filed on Sep. 14, 2001, the contents of both expressly incorporated herein by reference. At the end of processing, the concentrated cells may be loaded into a delivery device, such as a syringe, for placement into the recipient by any means known to one of ordinary skill in the art.

### Examples

Hereinafter, Examples of the present disclosure will be described. However, the present disclosure is not limited by these Examples.

### I: Test Subject Population

A total of 7 subjects of patients diagnosed with liver cirrhosis based on non-alcoholic steatohepatitis and patients diagnosed with liver cirrhosis based on fatty liver disease were tested.

Patients meeting all the following items were used as subjects.
(1) A patient diagnosed with liver cirrhosis based on (i) or (ii) by image findings or histology
   (i) Non-alcoholic steatohepatitis satisfying the following criteria:
      an alcohol intake is 20 g or less per day in terms of ethanol consumption;
      other known causes of liver damage have not been identified; and
      the patient has at least one possible condition or complication responsible for fatty liver, selected from the group consisting of obesity, visceral fat, metabolic syndrome and diabetes.
   (ii) Fatty liver disease satisfying the following criteria:
      an alcohol intake is more than 20 g and 70 g or less per day in terms of ethanol
      consumption;
      other known causes of liver damage have not been identified; and
      the patient has at least one possible condition or complication responsible for fatty liver, selected from the group consisting of obesity, visceral fat, metabolic syndrome and diabetes.
(2) A patient having prothrombin activity of 70% or more at the start of treatment
(3) A patient having a serum albumin level of 4.0 g/dL or less at the start of treatment
(4) A patient having a total bilirubin value of 3.0 mg/dL or less at the start of treatment
(5) A patient having a platelet count of 5.0×10⁴/μL or more at the start of treatment
(6) A patient having a serum creatine level 1.5 mg/dL or less at the start of treatment

### II: Collection of Adipose Tissue

An appropriately amount of a mixed solution containing 1,000 mL of physiological saline and 2 mL of a xylocaine injection “1%” with epinephrine (1:100,000) was injected, for distension, to subcutaneous adipose tissue in the buttocks or abdominal wall of each patient under general anesthesia or under local and lumbar anesthesia.

200 to 400 mL of the subcutaneous adipose tissue was collected. A 3 mm standard liposuction cannula with Mercedes tip was used in the collection.

### III: Separation of ADRCs

ADRCs were separated from the collected subcutaneous adipose tissue using a completely aseptic closed-type adipose tissue separation apparatus (Celution® 800/IV; Cytori Therapeutics Inc.), and a 5 mL cell suspension containing ADRCs in a lactated ringer's solution was recovered.

The number of cells and the percentage of live cells were measured using Nucleocounter (ChemoMetec A/S). The cell suspension containing ADRCs was confirmed to contain 3.3×10⁵ cells/kg×BW/5 mL or more and to have a live cell percentage of 70% or more.

### IV: Administration of ADRCs

The separated ADRCs were adjusted to 1×10⁶ cells/mL with a lactated ringer's solution, After insertion of the tip of a catheter (Microcatheter IV; Asahi Intecc Co., Ltd.) to the common hepatic artery from the femoral artery or the brachial artery, the cell suspension was administered in an amount corresponding to 3.3×10⁵ cells/kg over 30 minutes through the catheter.

The ADRCs used were freshly isolated cells without being cultured, and the ADRCs thus isolated were injected to patients on the same day as that of the isolation from the subcutaneous adipose tissue.

TABLE 1

| | | | | cell density | live cell | | |
|---|---|---|---|---|---|---|---|
| | | | amount of | of original | densityof | | amount of |
| | | | original cell | cell suspension | original | catheter | administrated |
| | | adipose tissue | suspension | after isolation | cell suspension | insertion | cell suspension |
| | Primary disease | harvest location | (mL) | (×10⁴/mL) | (×10⁴/mL) | site | (mL) |
| −1 | NASH | Abdominal wall | 5 | 1260 | 1191 | femoral artery | 27 |
| −2 | NASH | Buttocks/abdominal wall | 4.5 | 1071 | 963 | femoral artery | 20 |
| −3 | NASH | Abdominal wall | 5 | 1560 | 1460 | femoral artery | 28.3 |
| −4 | NASH | Abdominal wall | 5 | 2014 | 1764 | femoral artery | 26.04 |
| −5 | NASH | Abdominal wall | 5 | 792 | 732 | femoral artery | 18.5 |
| −7 | fatty liver disease | Abdominal wall | 5 | 2259 | 2115 | femoral artery | 34.7 |
| −8 | NASH | Abdominal wall | 5 | 2438 | 2214 | femoral artery | 30.8 |

### V: Characterization of ADRCs

The cell population containing ADRCs is performed by fluorescence activated cell sorting analysis before administration to patients, using a fluorescence labelled antibody against a cell membrane protein CD34, CD44, CD45. CD90 and CD105, and thus isolated ADRCs are characterized. The results are shown in Table 2.

TABLE 2

| item | category | descriptive statistics |
|---|---|---|
| (CD34) (%) | number of case | 7 |
| | average | 6.533 |
| | standard deviation | 7.2941 |
| | minimum | 0.76 |
| | median | 4.330 |
| | maximum | 22.00 |
| (CD44) (%) | number of case | 7 |
| | average | 65.986 |
| | standard deviation | 20.7903 |
| | minimum | 35.99 |
| | median | 73.290 |
| | maximum | 93.59 |
| (CD45) (%) | number of case | 7 |
| | average | 3.071 |
| | standard deviation | 2.9945 |
| | minimum | 0.73 |
| | median | 2.110 |
| | maximum | 9.32 |
| (CD90) (%) | number of case | 7 |
| | average | 6.571 |
| | standard deviation | 3.9425 |
| | minimum | 1.60 |
| | median | 5.850 |
| | maximum | 13.33 |
| (CD108) (%) | number of case | 7 |
| | average | 2.577 |
| | standard deviation | 2.9341 |
| | minimum | 0.00 |
| | median | 2.120 |
| | maximum | 8.32 |

Means from a total of 7 cases were 6.833±7.2941% for CD34, 66.986±20.7903% for CD44, 3.071±2.9945% for CD45, 6.571±3.9425% for CD90, and 2.577±2.9341% for CD105.

### VI: Liver Function Test

Acute and chronic hepatocellular injury can lead to an impairment of liver function (liver failure). Liver function tests are useful in investigating suspected liver disease, as well as monitoring disease activity, with abnormal values generally implying advanced liver disease. The common tests that assess liver synthetic function are serum albumin levels and prothrombin activity.

Low serum albumin is indicative of impaired hepatocellular function and associated with liver cirrhosis. Patients with cirrhosis have reduced albumin synthesis, which can reach a 60%-80% reduction in advanced cirrhosis. Protein levels further decrease due to the dilution effect from water and salt retention, and to the sequestration of circulating albumin in extracellular space and ascitic fluid. Importantly, serum albumin has prognostic significance, being a significant predictor of death in over a hundred studies in patients with cirrhosis. Serum albumin is a component of the most important and widely used prognostic score in cirrhosis, the Child-Pugh-Turcotte score.

Prothrombin time (PT) is a universal indicator of the severity of liver disease and is determined by vitamin K coagulation factors and fibrinogen. The liver produces the majority of coagulation proteins needed in blood clotting cascade. Severe liver injury leads to reduction of liver synthesis of clotting factors and consequently prolonged PT. PT is used in prognostic models of survival and is a key criterion for acute liver failure PT results are reported in seconds, as prothrombin ratios (PTR) expressed as percentages, and as international normalized ratios (INR).

As for the 7 patients, follow-up was performed over 24 weeks after administration of ADRCs, and the presence or absence of improvement in hepatic functions was evaluated on the basis of serum albumin levels and prothrombin activity. The results are shown below.

When the serum albumin level on 12 weeks after administration of ADRCs was elevated from the value obtained before administration, the serum albumin level was defined as having been improved. The FAS population was analyzed to evaluate efficacy. The rate of improvement in serum albumin level was calculated and tested on the basis of normal approximation with a threshold of 3% and a one-sided significance level of 5%.

The Clopper-Pearson method was used in calculation of confidence interval.

TABLE 3

| | | ratio test | 95% one-side |
|---|---|---|---|
| | number | (one-side normal | confidence interval |
| | of case | approximation *) | (Clopper- |
| item | (%) | statics | Pearson method) |
| improvement in | 6(85.7) | improvement | 47.9~99.3% |
| albumin level | | rate: 85.7(%) | |
| non-improvement in | 1(14.3) | p < 0.0001 | |
| albumin level | | | |
| total | 7(100.0) | | |

* Hₒ: p < 0.03

The rate of improvement in albumin level was 85.7% (95% confidence interval: 47.9 to 99.3%), and the test results were significant (p<0.0001).

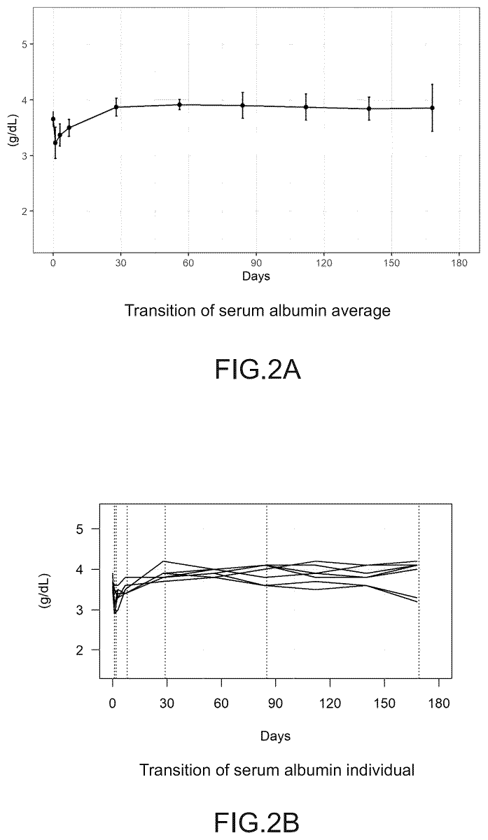

Changes in serum albumin level are shown in FIGS. 2A and 2B.

The changes in serum albumin level were 3.66±0.140 g/dL at baseline, 3.87±0.160 g/dL (p=0.0675) on 4 weeks after administration, 3.91±0.090 g/dL (p=0.0041) on 8 weeks after administration, 3.90±0.231 g/dL (p=0.0278) on 12 weeks after administration, 3.87±0.236 g/dL (p=0.0994) on 16 weeks after administration, 3.84±0.207 g/dL (p=0.0947) on 20 weeks after administration, and 3.86±0.420 g/dL (p=0.2467) on 24 weeks after administration, showing significant elevation on 8 and 12 weeks after administration.

Similarly, the rate of improvement was also calculated and evaluated as to prothrombin activity. The results are shown below.

TABLE 4

| | | ratio test | one-side |
|---|---|---|---|
| | number | (one-side normal | confidence interval |
| | of case | approximation *) | (Clopper- |
| item | (%) | statics | Pearson method) |
| improvement in | 5(71.4) | improvement | 34.1~94.7% |
| prothrombin activity | | rate: 71.4(%) | |
| non-improvement in | 2(28.6) | p < 0.0001 | |
| prothrombin activity | | | |
| total | 7(100.0) | | |

* Hₒ: p < 0.03

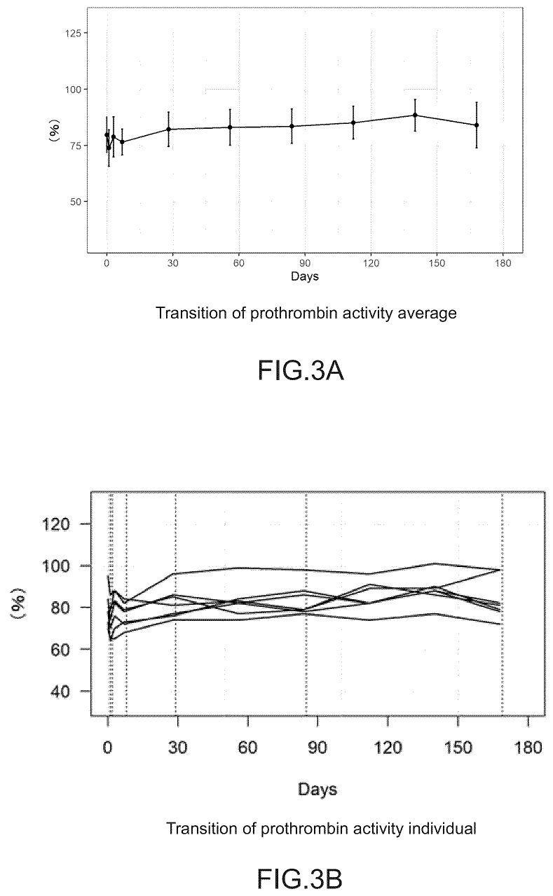

The rate of improvement in prothrombin activity was 71.4% (95% confidence interval: 34.1 to 94.7%), and the test results were significant (p<0.0001). Changes in prothrombin activity are shown in FIGS. 3A and 3B.

The changes in prothrombin activity were 79.7±8.14% at baseline, 82.1±7.60% (p=0.0179) on 4 weeks after administration, 83.0 t 7.92% (p=0.1126) on 8 weeks after administration, 83.6±7.63% (p=0.1156) on 12 weeks after administration, 85.1±7.31% (p=0.0297) on 16 weeks after administration, 88.4±7.04% (p=0.0019) on 20 weeks after administration, and 84.0 t 10.08% (p=0.2007) on 24 weeks after administration, showing significant elevation on 4, 16, and 20 weeks after administration.

### VII: Liver Biopsy

NAFLD activity score (NAS) and Matteoni classification according to liver biopsy are evaluated.

Histological examination of liver tissue specimens is the gold standard for quantitating steatosis, diagnosing nonalcoholic steatohepatitis (NASH) and staging fibrosis. Steatosis, namely a minimal threshold of 5% of hepatocytes containing fat droplets in biopsy specimen, is a prerequisite for the diagnosis of nonalcoholic fatty liver disease (NAFLD). NAFLD may manifest histologically as nonalcoholic fatty liver (NAFL) or NASH. Whereas NAFL is defined as the presence of hepatic steatosis with no evidence of hepatocellular injury (ballooning of the hepatocytes), NASH comprises the presence of a combination of hepatic steatosis, lobular inflammation and hepatocellular ballooning, which are believed to be the primary drivers of fibrogenesis that ultimately lead to progressive fibrosis and cirrhosis.

Matteoni et al. presented the first diagnostic criteria to categorize NAFLD into four different subtypes: NAFLD type 1 with fatty liver alone; type 2 with fatty liver plus lobular inflammation; type 3 with fatty liver plus ballooning degeneration; and type 4 with fat accumulation, ballooning degeneration and either Mallory-Denk bodies or fibrosis (Matteoni et al., Nonalcoholic fatty liver disease: a spectrum of clinical and pathological severity. Gastroenterology. 1999 June; 16(6):1413-9). There was a trend for increased liver-related mortality in patients with subtypes 3 and 4 compared with subtypes 1 and 2. The subtypes 3 and 4 are those that we consider today to represent NASH. On the other hand, NAS, an unweighted sum ranging from 0 to 8 (with 8 indicating more severe disease), consisting of 3 independent histological components: steatosis (0-3), lobular inflammation (0-3) and ballooning degeneration (0-2) (Kleiner et al., Nonalcoholic Steatohepatitis Clinical Research Network. Association of Histologic Disease Activity With Progression of Nonalcoholic Fatty Liver Disease. JAMA Netw Open. 2019 Oct. 2; 2(10): e1912565).

NAS includes features of active injury that are potentially reversible and was designed to evaluate the histological effect of interventional and therapeutic strategies. Currently, NAS is the most widely used histological classification for NAFLD/NASH and its use is recommended as an endpoint in clinical trials for defining and quantifying disease activity in interventional studies. In a study by Kleiner et al, an improvement in NAS and disease activity was associated with an improvement in fibrosis, and vice versa. The results of the study provide a rationale for the use of NAS as a surrogate end point in the short term in clinical trials of NASH using agents that improve disease activity such as described in the present disclosure.

Improvement in NAS (lobular inflammation) in 2 cases and improvement in NAS (steatosis) and fibrosis in 1 case were found on 24 weeks after administration.

Improvement in NAS (hepatocyte ballooning) were found in a large proportion of patients on 24 weeks after administration.

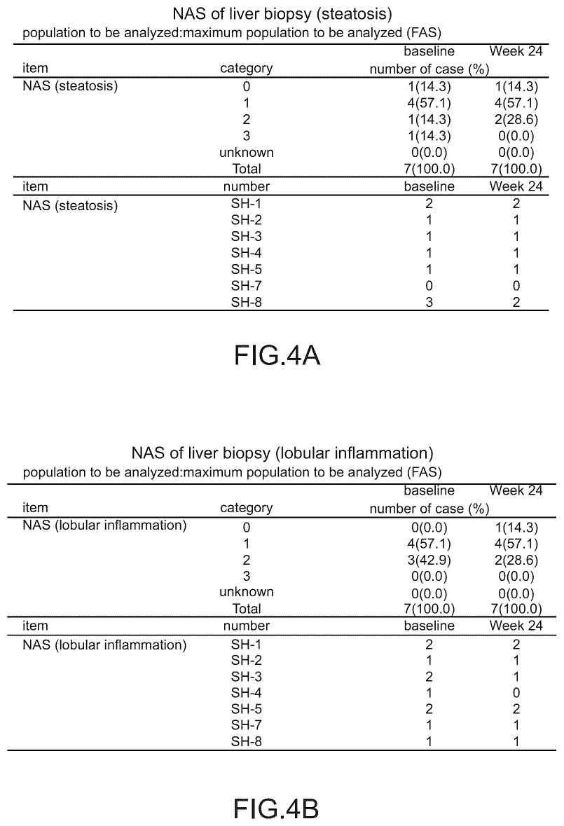

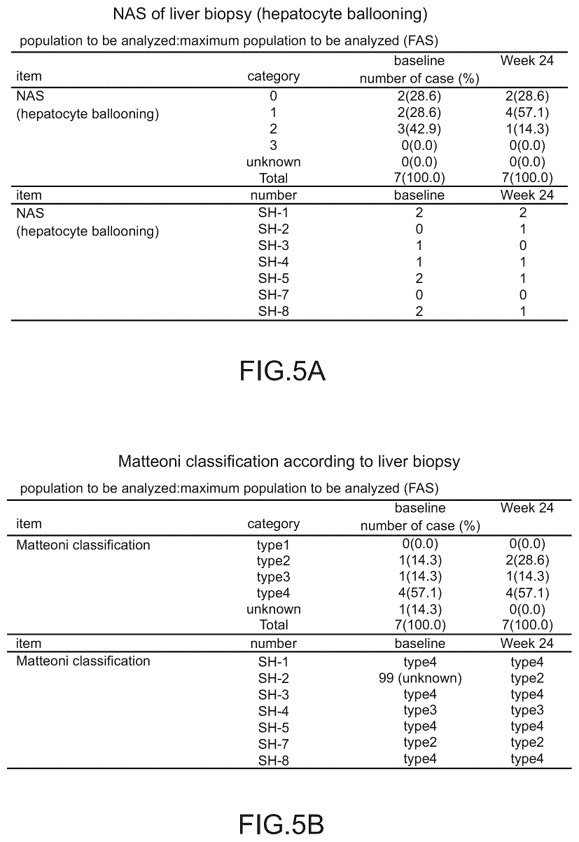

No change in the Matteoni classification was found on 24 weeks after administration. The results are shown in FIGS. 4A, 4B, 5A and 5B.

### VIII: Conclusion

Improvement both in serum albumin level and in prothrombin activity were found in 5 out of the 7 liver cirrhosis patients. Cases with improvement in liver condition were also found in liver histology by liver biopsy. The test results described above indicate that treatment using ARDCs, particularly, freshly isolated autologous ADRCs, are effective for improvement in or maintenance of hepatic functions in a patient diagnosed with liver cirrhosis based on non-alcoholic steatohepatitis (NASH) or a patient diagnosed with liver cirrhosis based on fatty liver disease.

Especially, from the above results, it can be said that the cells described herein, e.g., adipose-derived cells including stem cells, adipose-derived cells including regenerative cells, adipose-derived cells including stem and regenerative cells, and the like, a heterogeneous mix of living cells capable of changing their transcriptome and secretome in response to physiologic stimuli, have the ability to act on liver regeneration (by treating or preventing fibrosis) through a multiplicity of actions. It is anticipated that the cellular cross talk between the various cell lineages and multiplicity of factors released will have additive and synergistic effects such that the outcome would not have been possible by released of just one or two of these factors. Exemplary mechanisms regulated by the microenvironment during liver regeneration include but are not limited to angiogenesis, anti-fibrosis and remodeling, and others.

ADRCs have been shown to be capable of promoting angiogenesis by both the presence of endothelial progenitor cells (EPC) and by expression of pro-angiogenic factors. EPCs have been shown to improve survival and hepatic function in animal models of liver disease. It is likely that EPCs act, at least in part, by promoting new vessel formation and improving hepatocyte perfusion. Liver cirrhosis involves establishment of intrahepatic vascular shunts that can dramatically reduce hepatocyte perfusion and create a hypoxic environment that is hostile to regeneration and repair. Indeed, the development of extensive intrahepatic shunts has been described as the major determinant of the point of no return for cirrhosis. EPCs likely act by improving the microenvironment within the liver through formation of new vessels and extracellular matrix remodeling. This creates a microenvironment in which normal liver repair mechanism are able to operate more effectively as evidenced by increased hepatocyte proliferation in animals treated with EPCs. Thus, approaches that increase hepatic angiogenesis have the potential to promote improved hepatic regeneration.

Moreover, the hepatocyte growth factor (HGF) is a well-known anti-fibrotic cytokine, and delivery of the HGF gene or protein attenuates liver fibrosis in numerous in vivo models. The anti-fibrotic effect of HGF is thought to be achieved through attenuation of fibrogenic cytokine expression (transforming growth factor beta 1 (TGF-β1) and platelet-derived growth factor-bb (PDGF-bb)), and through inhibition of the proliferation and activation of hepatic stellate cells, the major extracellular matrix producer in the liver. In addition, HGF inhibits the cell death of normal hepatocyte. ADRCs have been shown to secrete HGF, at significantly higher levels compared to mesenchymal stem cells, including adipose derived mesenchymal stem cells. It is expected the use of ADRCs via the secretion of various growth factors and cytokines such as HGF, will be therapeutically effective in promoting recovery from fibrosis and improvement in hepatocyte function.

1. A pharmaceutical composition for use in prevention or treatment of liver fibrosis or liver cirrhosis, comprising adipose tissue-derived regenerative cells (ADRCs).

2. The pharmaceutical composition according to claim 1, wherein the ADRCs are an arbitrary heterogeneous or homogeneous cell population containing one or more types of adipose-derived regenerative cells selected from adipose-derived stem cells (ADSCs), endothelial cells, endothelial precursor cells, macrophages, fibroblasts, pericytes, smooth muscle cells, preadipocytes, keratinocytes, unipotent or multipotent precursor and progenitor cells and progeny thereof, and lymphocytes.

3. The pharmaceutical composition according to claim 1, wherein the adipose-derived regenerative cells comprise ADRCs at a proportion of at least 0.1% of all cellular components of the ADRCs.

4. The pharmaceutical composition according to claim 1, wherein the ADRCs are uncultured cells.

5. The pharmaceutical composition according to claim 1, wherein the ADRCs are cryopreserved cells.

6. The pharmaceutical composition according to claim 1, wherein the ADRCs are autologous subcutaneous adipose tissue-derived regenerative cells.

7. The pharmaceutical composition according to claim 1, wherein the ADRCs are positive for a cell surface marker of CD45.

8. The pharmaceutical composition according to claim 1, wherein the liver fibrosis and/or liver cirrhosis is caused by a chronic liver injury.

9. The pharmaceutical composition according to claim 1, wherein the liver fibrosis and/or liver cirrhosis is caused by non-alcoholic steatohepatitis.

10. The pharmaceutical composition according to claim 9 for application to a patient diagnosed with liver cirrhosis caused by non-alcoholic steatohepatitis by image findings or histology, the patient satisfying the following criteria (1) to (3):
(1) an alcohol intake is 20 g or less per day in terms of ethanol consumption;
(2) other known causes of liver damage have not been identified; and
(3) the patient has at least one possible condition or complication responsible for fatty liver, selected from the group consisting of obesity, visceral fat, metabolic syndrome and diabetes.

11. The pharmaceutical composition according to claim 1, wherein the liver fibrosis and/or liver cirrhosis is based on fatty liver disease.

12. The pharmaceutical composition according to claim 11 for application to a patient diagnosed with liver cirrhosis based on fatty liver disease by image findings or histology, the patient satisfying the following criteria (4) to (6):
(4) an alcohol intake is more than 20 g and 70 g or less per day in terms of ethanol consumption;
(5) other known causes of liver damage have not been identified; and
(6) the patient has at least one possible condition or complication responsible for fatty liver, selected from the group consisting of obesity, visceral fat, metabolic syndrome and diabetes.

13. The pharmaceutical composition according claim 1, wherein the pharmaceutical composition is prepared such that the pharmaceutical composition is administered by intra-arterial, intravenous or intraportal injection.

14. The pharmaceutical composition according to claim 1, wherein the pharmaceutical composition is prepared in an amount of a cell density of 1×10³ to 1×10⁹ cells/mL.

15. The pharmaceutical composition according claim 1, wherein the treatment of the liver fibrosis and/or the liver cirrhosis comprises improvement in serum albumin level and/or prothrombin activity.

16. The pharmaceutical composition according claim 1, wherein the pharmaceutical composition is prepared in the form of a single-cell suspension of ADRCs.
